(19) United States
(12) Patent Application Publication
(10) Pub. No.: US 2002/0037560 A1
(43) Pub. Date: Mar. 28, 2002

(54) Bicyclic terpenes and synthesis thereof

(76) Inventors: Joseph Chappell, Lexington, KY (US); H.P. Spielmann, Lexington, KY (US)

Correspondence Address: MCDERMOTT WILL & EMERY, 600 13TH STREET, N.W., WASHINGTON, DC, 20005-3096, US

(21) Appl. No.: 09/876,169

(22) Filed: Jun. 8, 2001

Related U.S. Application Data
(60) Provisional application No. 60/210,527, filed on Jun. 9, 2000.

(51) Int. Cl. C12P 17/10; C07D 487/00

(52) U.S. Cl. 435/121; 540/477

(57) ABSTRACT

Thirteen-membered ring containing terpenoid analog compounds are synthesized from analinogeranylpyrophosphate using 5-epi-aristolochene synthase as a reaction catalyst. The method provides a generalized procedure for making high-ordered ring structures having various substituent groups. The products can be used in assays for 5-epi-aristolochene synthase activity, and as precursors and intermediates for biologically active substances.

### CROSS-REFERENCE TO RELATED APPLICATIONS

This application claims priority from U.S. provisional application serial no. 60/210,527, filed on Jun. 9, 2000.

### FIELD OF THE INVENTION

The present invention relates to compounds having a unique bicyclic ring structure and to methods of synthesizing such compounds.

### BACKGROUND OF THE INVENTION

Synthetic approaches to five- and six-membered ring systems are legion, encompassing both cyclization and cycloaddition approaches. But seven- to fifteen-membered (and larger) ring systems are very difficult to achieve. Cyclization strategies for synthesizing medium- and larger-sized rings are often regarded as inappropriate because of entropic factors that impede ring closure.

Although a significant number of synthetic methods have proven valuable for the construction of medium-sized carbocyclic and heterocyclic systems, there are limitations on the range of chemical functionalities, and ring structures obtained. The classical approaches to medium- to large-ring molecules fall into three different categories: ring expansion from smaller cyclic units, cyclization methods, in which a single bond is created in the key step to construct the medium-sized ring, and annulative approaches in which two acyclic precursors are brought together to generate the cyclic unit with formation of two bonds in a one-pot reaction.

Normally, simple cyclization routes to medium-membered rings result in low yields of the desired products. Because of entropic factors, the coupling of two reactive termini spaced some distance apart is quite difficult. In the formation of medium- to large-membered rings, trans-annular interactions that develop among the intervening atoms cooperate to make undesired intermolecular coupling much more facile than cyclization. From a synthetic point of view, it is important that regiochemistry and stereochemistry be controlled in the formation of medium and large rings.

To date none of the above-identified approaches has proven suitable for making a bicyclic compound having a 13-membered ring as disclosed below.

### SUMMARY OF THE INVENTION

The foregoing and other needs are met by embodiments according to the present invention, which provide a compound of the formula:

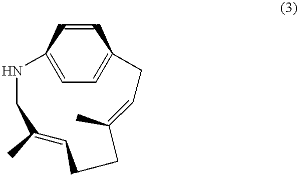

and salts and derivatives thereof.

The foregoing and other needs are met by embodiments according to the present invention, which provide a method of making a compound of formula:

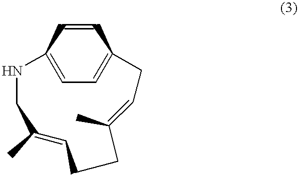

said method comprising cyclizing a compound of the formula:

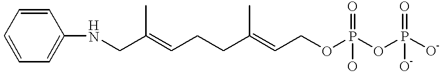

in the presence of an enzyme to form the inventive compound (3).

### BRIEF DESCRIPTION OF THE DRAWING(S)

FIG. 1 is a Lineweaver-Burk plot showing the kinetic characterization of the interaction of AGPP (8-anilino-geranyl pyrophosphate) with TEAS.

FIG. 2 is an electron density map of AGPP-TEAS.

FIG. 3 is a two-dimensional representation of the tertiary structure of TEAS.

FIG. 4 is a two-dimensional close-up representation of the putative active site of TEAS.

FIG. 5 is a gas chromatogram of hexane extracts from reactions containing AGPP and TEAS.

FIG. 6a is a gas chromatogram of a 5-epi-aristolochene standard.

FIG. 6b is high resolution mass spectrogram of a 5-epi-aristolochene standard.

FIG. 7a is a gas chromatogram of hexane extracts from reactions containing AGPP and TEAS.

FIG. 7b is high resolution mass spectrogram of hexane extracts from reactions containing AGPP and TEAS.

### DETAILED DESCRIPTION OF THE INVENTION

The present invention relies on a number of key observations concerning the flexibility of terpene synthases to utilize synthetic substrates for the biosynthesis of new chemical compounds. These types of compounds have utility in industrial, medicinal, biotechnological and agricultural applications. As an example of the inventive compounds' biotechnological utility, the compounds may be used as assay reagents in Tobacco 5-epi-aristolochene synthase (TEAS) binding assays. Organic synthesis of these types of compounds has heretofore proven difficult. The compounds are complex, and would normally require concomitantly complex organic synthesis schemes. In some cases, current organic synthesis techniques are not sufficiently developed to yield these types of products. Yields from any synthetic efforts are also likely to be quite low. Most importantly, conventional synthetic means cannot afford regio- and stero-chemical purity of the resulting compounds. It is exactly these properties of the resulting compounds, their regio- and stereo-chemistry, which directly translates into the value of these compounds for practical applications.

The cyclization of AGPP by Tobacco 5-epi-aristolochene synthase (TEAS) falls into the category of cyclization reactions. While not wishing to be bound by any theory, it is believed that the cyclization of AGPP to form the inventive compounds of formula (3) proceeds by the synthetic pathway set forth in Scheme 2, below. As shown in Scheme 2, the first step after AGPP binds to the TEAS active site is extraction of the pyrophosphate moiety ⁻ OPP:

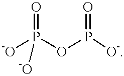

In the next step, the para-position carbon of the benzene ring attacks the positively charged α-carbon of the geranyl group, with loss of the para-position proton to form the target product (3).

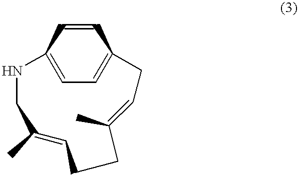

The inventive compound (3) may be prepared in the form of a salt. In particular the cyclic amine group in compound (3) may react with an acid to form an acid addition salt. Suitable acids for forming acid addition salts include inorganic acids such as hydrohalic acids (e.g. hydrochloric and hydrobromic acid), phosphoric acid, carbonic acid, sulfuric acid, boric acid, etc. Other suitable acids for forming acid addition salts include organic acids such as formic acid, acetic acid, and benzoic acid. In general, the acids are added to (3) in stoichiometric equivalent or slight stoichiometric excess to form the desired salt.

The inventive compound (3) may be derivatized by a suitable prior art method for forming derivatives, especially of a cyclic amine or an aromatic compound. In particular, the cyclic amine may be derivatized to form an amide by reacting the amine of (3) with a carboxylic acid, acid anhydride or acid halide under conditions suitable to make an amide. The cyclic amine may also be derivatized to form a sulfonamide by reacting the compound (3) with a suitable sulfonic acid or sulfonyl halide under suitable conditions. The reaction may be driven toward the amide or sulfonamide side by removal of water during the reaction, for instance by a desiccant or by heating under reflux conditions. Suitable carboxylic acids include C₁-C₆ alkanoic acids, C₁-C₆ alkenoic acids, and aryl-C₁-C₆-alkanoic acids, such as acetic acid, propionic acid, benzoic acid, and derivatives thereof. Suitable sulfonic acids include C₁-C₆-alkylsulfonic acids, C₁-C₆alkenylsulfonic acids, and aryl-C₁-C₆-alkanesulfonic acids.

The benzene ring of (3) may also be derivatized, for instance by subjecting the ring to conditions suitable for placing a conventional substituent on the ring. For instance, halide substituents may be placed on the ring by reacting (3) with suitable halidation reagents (such as Br₂ in the presence of ultraviolet (UV) light). Alkyl and acyl groups may be added to the ring by Friedel-Crafts addition, using alkylhalides, aralkylhalides, alkanoylhalides, or aralkanoylhalides as reagents.

The preferred embodiments according to the present invention will now illustrated by the following examples, which are intended to be illustrative, not limiting, of the present invention. In particular, while the examples specifically employ TEAS, other enzymes may also be employed, without departing from the scope of the present invention. In some embodiments according to the present invention, 5-epi-aristolochene synthases derived from plant species other than tobacco, and in particular other members of the genus nicotiana, may be employed. The skilled artisan will recognize that as compound (3) is a competitive inhibitor of TEAS, a suitable 5-epi-aristolochene synthase may be isolated by conventional methods, using compound (3) as an assay reagent for detecting and measuring 5-epi-aristolochene synthase activity. In this regard, a means for cyclyzing AGPP includes all 5-epi-aristolochene synthases from whatever source derived, and especially those 5-epi-aristolochene synthases derived from the genus nicotiana, particularly those that are competitively inhibited by compound (3), a salt or derivative thereof.

### EXAMPLES

### Materials and Methods

Materials. [1-³H]FPP (1) (20.5-21.5 Ci/mmol) was purchased from DuPont-NEN. 8-anilino-geranyl pyrophosphate (AGPP; (2)), 8,8-³H-anilino-geranyl pyrophosphate ([³H]AGPP, (22)), 8-anilinogeraniol (AGOH) were synthesized according to published procedures (Chehade et al.) and were stored at −20° C. AGPP for kinetic and GC-MS experiments was stored as a 2 mM solution in water. AGPP for crystallization experiments was stored as a solid and was solubilized as needed. [³H]AGPP (17 Ci/mmol) was stored at 8 μCi/μL in water. The AGOH stock was 100 mg/μL in methanol. A 5-epi-aristolochene GC-MS standard was a gift from Robert Coates (Department of Chemistry, University of Illinois, Urbana-Champaign). the 5-epi-aristolochene was generated enzymatically from FPP, using TEAS provided by Joseph Chappell (Department of Agronomy, University of Kentucky), and was purified to approximately 90% according to GC analysis, the structure of the enzymatic reaction product was confirmed by NOE NMR. Unless indicated otherwise, all other chemicals were from Sigma or Fisher.

Bacterial Expression and Purification of Recombinant TEAS for Kinetic and GC-MS Experiments. The expression and purification of TEAS for kinetic and GC-MS experiments were based on previously published procedures (Mathis et al., 1997), with modifications as described. Cells collected from 300 ml of culture were resuspended in as minimal volume of Buffer A (500 mM NaCl, 20 mM Tris-Cl, pH 7.9), frozen overnight at −80° C., then thawed at room temperature. Additional Buffer A was added to a maximum volume of approximately 12 ml and the suspension was supplemented with 1 mg/ml lysozyme. Following incubation on ice for 30 min., the cells were disrupted by sonication (3×30 second pulses) and the lysate was clarified by centrifugation at 39000 g for 20 min. The supernatant, containing TEAS, was filtered (0.45 μm) and applied to a 2 ml column of His-Bind Ni²⁺-affinity resin (Novagen), equilibrated in Buffer A at a flow rate of approximately 20 ml/hr. The column was washed with 20 ml of equilibration buffer and TEAS was eluted with a 20 ml linear gradient to 250 mM imidazole in this buffer. Fractions were assayed for protein according to Bradford (1976), using the Bio-Rad reagent. The protein peak was dialyzed against Buffer B (50 mM HEPES, pH 7.5, 5 mM MgCl₂) containing 1 mM DTT, and then concentrated to approximately 8 mg/ml using a centrifugal filter unit (Millipore Ultrafree-4 Biomax-30; 30 kDa MWCO). Glycerol was added to 50%, and the protein was stored at −80° C. SDS-PAGE analysis of a typical preparation indicated a TEAS purity of >80%.

In some cases, TEAS was purified further by anion exchange chromatography on a MonoQ HR5/5 column (Pharmacia), equilibrated in Buffer B at a flow rate of 1 ml/min. Following protein application, the column was washed with 10 ml of equilibration buffer and TEAS was eluted with a 30 ml linear gradient to 0.3 M NaCl in this buffer. The protein peak containing TEAS activity (details of the TEAS activity assay are presented below) was dialyzed and concentrated to 20-40 mg/ml, as described above. Glycerol was added to 50%, and the protein was stored as described. SDS-PAGE analysis of a typical extended TEAS purification indicated at TEAS purity of >95%.

Bacterial Expression and Purification of Recombinant TEAS for Crystallization Experiments. The expression and purification of TEAS for crystallization experiments also were based on previously published procedures (Mathis et al., 1997), with modifications as described. TEAS expression was induced in 4 L of cells, which were then incubated for 15-20 hrs. at 22° C. The cells were collected and resuspended in 5 ml of Buffer C (20 mM imidazole, 500 mM NaCl, 20 mM Tris-Cl, pH 7.9) per g of cells (weight/weight), and stirred for 30 min. at 4° C. The cells were lysed by sonication and the lysate was clarified by centrifugation at 82000 g for 40 min. The supernatant was loaded onto a 2-3 ml Ni²⁺-affinity column (Qiagen), equilibrated in Buffer C, and the column was washed with additional Buffer C until the A₂₈₀ of the eluate returned to baseline. TEAS was eluted with a linear gradient to 200 mM imidazole in Buffer C. Fractions containing protein were pooled and dialyzed against Buffer B containing 1 mM DTT.

TEAS was then applied to a MonoQ HR 10/10 cation exchange column (Pharmacia), equilibrated in Buffer B containing 1 mM DTT. The column was washed with 20 volumes of equilibration buffer and TEAS was eluted with a linear gradient of 0.5 M NaCl, 2 mM MgCl₂, 50 mM Tris-Cl, pH 8.0. Purified TEAS was dialyzed against 5 mM NaCl, 1 mM DTT, 5 mM Tris-Cl, pH 8.0, concentrated to 18-22 ml, and stored at −80° C.

Kinetic Characterization of the Interaction of AGPP with TEAS. The assay for TEAS activity is based upon partitioning of the hydrophobic product (³H-5-epi-aristolochene) into an organic solvent while the hydrophilic substrate (³H-FPP) remains in the aqueous phase (i.e. Vögeli & Chappell, 1988; Back et al., 1994; Mathis et al. 1997). The Kᵢ describing AGPP interaction with TEAS was characterized by measuring initial velocities for a matrix of reactions. Reactions (50 μl) containing 200 mM Tris-Cl, pH 7.5, 40 mM MgCl₂, 50-250 nM TEAS, and 0.2 μCi ³H-FPP, carrier-free or in combination with unlabeled FPP to give final substrate concentrations of 0.2-10 μM. Fixed concentrations of AGPP ranged from 0-150 μM. Reactions were initiated by the addition of enzyme and were incubated for 5 min. at 37° C. Reactions were quenched by vortexing against 150 μl hexane. After a brief centrifugation, 100 μl of the hexane phase was treated with approximately 20 mg of silica powder to remove any contaminating FPP or farnesol, the latter of which may be generated by contaminating phosphatase activity. Following vortexing and a brief centrifugation to pellet the silica, 50 μl of the hexane phase was mixed with 4 ml of liquid scintillation cocktail and analyzed for radioactivity (disintegrations per minute). Determination of reaction rate was based on percent conversion to product, the value for which was obtained by comparing the radioactivity in the hexane extract to that in an untreated aliquot of the assay mixture. Near-background levels of radioactivity were observed in hexane extracts derived from control reactions lacking enzyme. In addition, silica treatment did not significantly alter the amount of radioactivity observed in hexane extracts, regardless of teas purity level, indicating insignificant phosphatase contamination. Data were analyzed using Enzyme Kinetics V 1.5 (Trinity Software).

Crystallization and Data Collection for TEAS-AGPP Complex. TEAS crystallizes in hanging drops (Starks et al., 1997). Crystals may grow as large as 0.3-0.4 mm on an edge, but smaller crystals (0.2 mm) give higher quality diffraction, possibly due to more homogeneous freezing throughout the crystal. For structure determination of TEAS-AGPP complex, a 0.2 mm TEAS crystal was soaked in mother liquor containing 1 mM AGPP, then stabilized for freezing in a similar solution which also included 20% ethylene glycol. The TEAS-AGPP crystal was frozen in a nitrogen stream (˜190° K.) and a diffraction data set was collected at Stanford Synchrotron Radiation Laboratory beamline 7-1 (Table 1).

TEAS-AGPP Structure Determination and Refinement. A starting model consisting of protein residues 17-522 and 533-548 of the TEAS-farnesyl hydroxyphosphonate structure (PBD code 5EAT) was positioned with respect to the TEAS-AGPP data using rigid body refinement in XPLOR (3.851, ref. The initial 3f₀-2F₀ difference electron density map revealed additional density for protein residues 523-532 as well as a molecule bound in the active site. the missing regions of the protein were built, water molecules were added, and several rounds of positional and temperature factor refinement and manual model adjustment were carried out. An energy-minimized (Chem3D) model of a putative product of TEAS catalysis with AGPP (3) was placed in the active site electron density, aided by the clear density for its phenyl and methyl groups. Additional refinement of the model, including (3), resulted in an R_{free} of 26.7% (Table 1). Model building was carried out with the program O (ref). Refinement, map calculation, and water molecule location were carried out with XPLOR and CNS (ref). Final coordinates of the TEAS-(3) complex have been submitted to the Protein Data Bank (accession code x).

Gas Chromatographic and Mass Spectrometric Analysis of the Hexane-Extractable Product of TEAS Incubation with AGPP. TEAS control reactions with the natural substrate contained 23 μM FPP, 40 mM MgCl₂, 200 mM Tris-Cl, pH 7.5, and 6 μM, TEAS in volume of 100 μl. Reactions were incubated for 30 min at 37° C. and were pooled into 500 μl aliquots that were extracted with 200 μl of hexane each (Fisher, standard grade). TEAS reactions with AGPP contained 200 μM AGPP, 40 μM MgCl₂, 10% glycerol, 200 mM Tris-Cl, pH 7.5 and 10 μM TEAS in a volume of 100 μl. Reactions were incubated at 4° C. for a period of 30 min. to several days, or at 30° C. for 30 min. Reactions were extracted twice with a total of 100 μl of hexane (Fisher Optima, GC-MS grade). Relatively low temperatures were generally used for incubations of TEAS with AGPP in an effort to approximate conditions used in parallel co-crystallization experiments.

Hexane extracts were analyzed by GC using a Hewlett-Packard 5890 Series II gas chromatograph, equipped with a flame-ionization detector and capillary DB5 column with He as the carrier gas (15 ml/min). Samples were introduced by (splitless) injection at 220° C. The column temperature was maintained at 60° C. for 30 seconds and was then increased to 280° C. with gradient of 10° C. per min.

Hexane extracts of TEAS reactions with AGPP were prepared for GC-MS analysis essentially as described for GC analysis. Reactions were incubated at 4° C. for several days and then pooled into 500 μl aliquots that were extracted twice with a total of 400-700 μl hexane each. For high resolution MS experiments, the pooled hexane extracts were concentrated to a minimal volume under N₂. The concentration of hexane-extractable product (0.1-3 μg/ml) was estimated by GC, based on comparison of peak area to that of a hexadecane standard.

The hexane extracts, and the 5-epi-aristolochene standard, were subjected to GC-MS analysis using a Varian 3400 gas chromatograph and a Finnigan INCOS-50 quadrupole mass selective detector. The GC was equipped with a capillary DAB-5MS column (15 m×0.25 mm, 0.25 μm phase thickness), with He as the carrier gas (10 psi). Samples of the hexane extracts were introduced by splitless injection at an injection port temperature of (210° C.). The column temperature was maintained at 60° C. for 1 min. following injection and was then increased to 280° C. with a gradient of 10° C. per min. GC-MS analysis of the 5-epi-aristolochene standard included splitless injection at an injection port temperature of (100 or 120° C.). The column temperature was maintained at 50° C. for 1 min. and then increased to 150° C. with a gradient of 4° C. per min. All samples were introduced directly to the electron impact ionization source for mass spectral analysis. Spectra were recorded at 70 eV, scanning from 20 to 420 atomic mass units. Mass spectral data were compared to those published for 5-epi-aristolochene (Whitehead et al., 1989).

The exact molecular mass of the hexane-extractable product of AGPP incubation with TEAS was determined by high resolution mass spectrometry, using a Kratos CONCEPT IH magnetic sector mass selective detector. Samples were introduced directly to the electron impact inonization source and each measurement was recorded at 70 eV. For each spectrum, approximately 20-30 scans were collected in a slow scanning mode (10 second/decade). The column bleed was used as the internal standard and was calibrated separately against perfluorokerosene. Reported data are based on a composite of the early strong scans for which an optimal response from the molecular ion was obtained.

### Results and Discussion

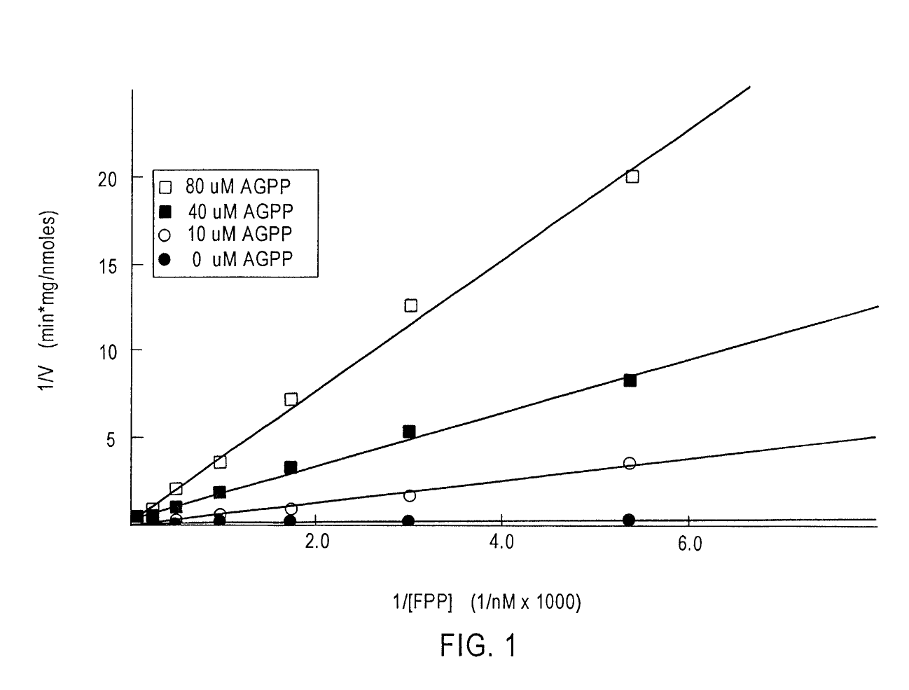

Kinetic Characterization of the Interaction of AGPP with TEAS. As a synthetic analog of FPP, AGPP (2) may potentially bind within the TEAS active site and thereby inhibit the sesquiterpene synthase activity of this enzyme. The nature of the potential interaction between TEAS and AGPP was characterized through a series of TEAS activity assays in which the ability of TEAS to produce ³H-5-epi-aristolochene from ³H-FPP was monitored in the presence of unlabeled AGPP. The pattern of intersecting Lineweaver-Burk plots obtained (FIG. 1) indicates that AGPP is a competitive inhibitor of TEAS-catalyzed cyclization of FPP and therefore binds within the TEAS active site. The Kᵢ for the inhibition of TEAS activity by AGPP, 5.9±1.8 μM, is very similar to the K_{M} of 5.4±μM that characterizes TEAS utilization of FPP as a substrate at 37° C. Thus, AGPP appears to be an effective ground state analog of FPP.

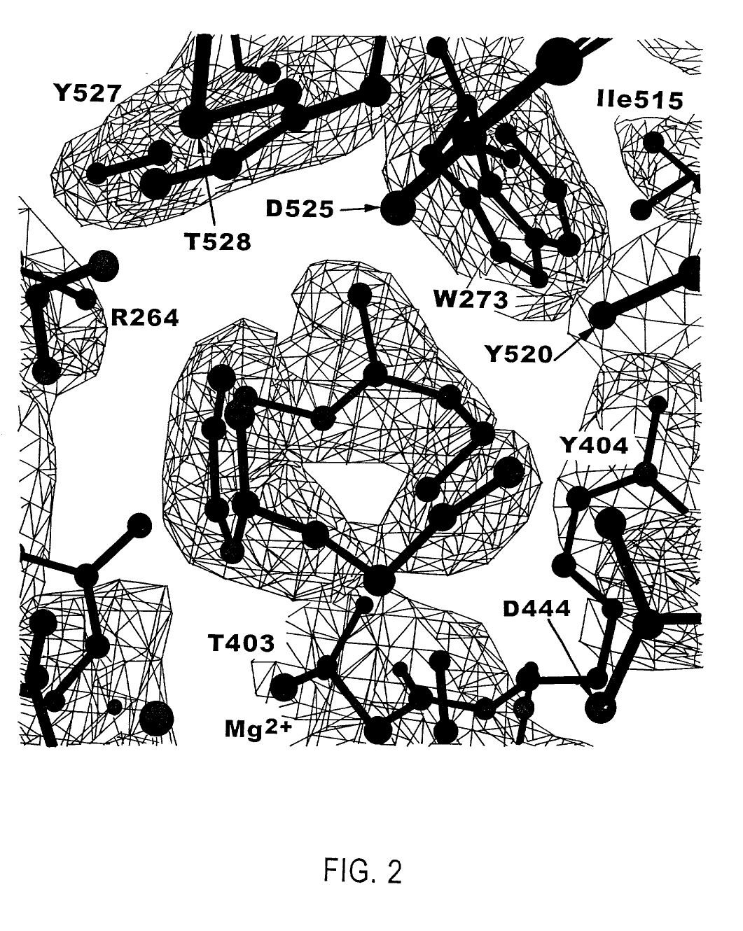

TEAS-AGPP Crystal Structure. Diffraction data were collected from TEAS crystal soaked in 1 mM AGPP, a concentration well above the Kᵢ for AGPP inhibition of TEAS catalysis. The initial 3f₀-f2f₀ difference electron density map exhibited a donut-shaped region of electron density in the TEAS active site (FIG. 2). The strong electron density expected for the pyrophosphate moiety of AGPP was not visible. The crystallographic evidence thus suggested that crystalline TEAS had catalyzed the cyclization of AGPP, concomitant with the ionization of pyrophosphate. An energy-minimized (Chem3D) model of macrocyclic alkaloid (C₁₆H₂₁N; (3)) was easily placed into the TEAS active site, with features in the ring of electron density indicating the locations of the phenyl and methyl groups. If the proposed AGPP cyclization occurs in analogy to TEAS-catalyzed FPP cyclization (Scheme 1), TEAS may promote the ionization of pyrophosphate from AGPP to form an allylic carbocation (Scheme 2). Since the amino substituent of the aniline ring activates the ortho and para positions of the phenyl moiety through resonance donation of pi electrons, electrophilic attack by the allylic carbocation would most likely occur at the less hindered para position. Loss of a proton to the enzyme or solvent yields the macrocyclic alkaloid (3).

Cyclization of FPP by TEAS is shown in Scheme 1, below.

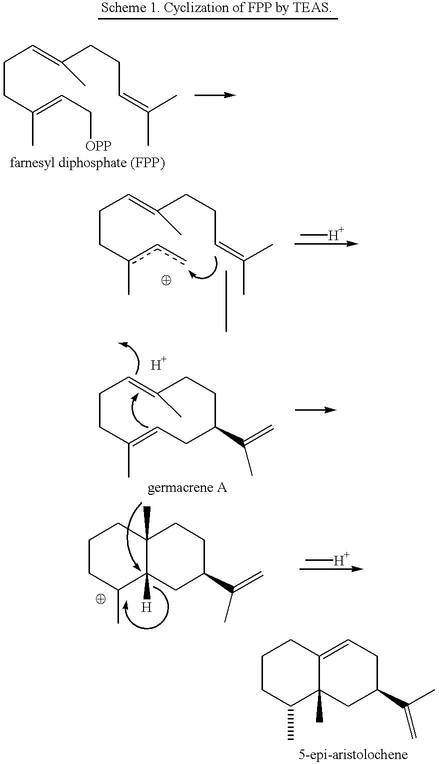

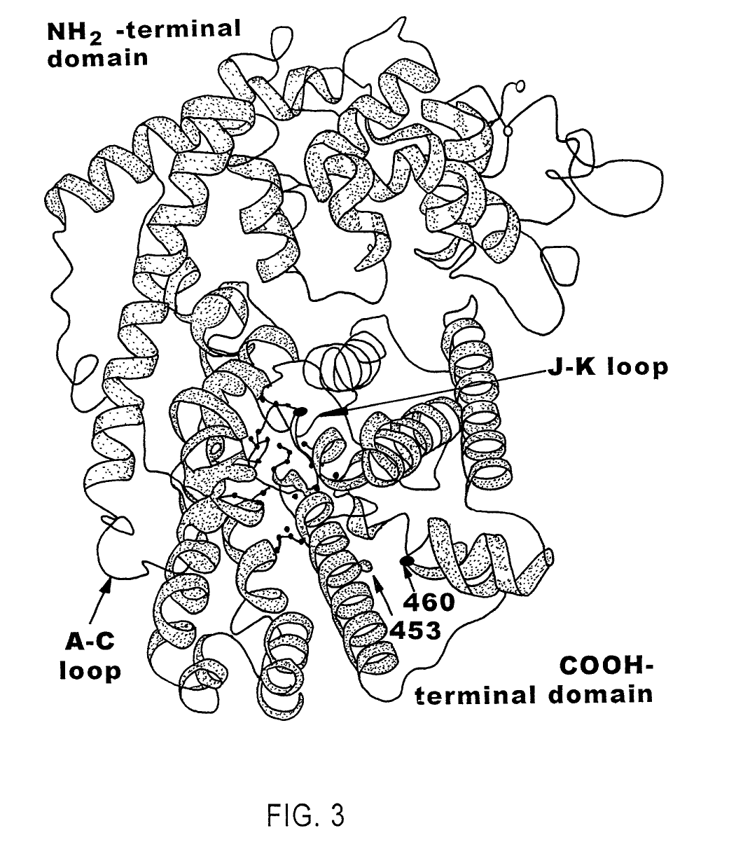

Several rounds of model building and refinement resulted in a model containing protein residues 19-97, 102-453, and 460-548, 152 water molecules, 1 magnesium ion and the macrocyclic alkaloid (3). Refinement statistics are summarized in Table 1. The overall structure of the TEAS-(3) complex is similar to the liganded and unliganded TEAS structures reported previously (Starks et al., 1997). The enzyme has two structural domains, both of which consist entirely of α-helices and connecting loops. The single active site is a deep, hydrophobic, aromatic-rich pocket in the C-terminal domain, with bound magnesium ions and positively charged residues at its entrance. In the absence of substrate analog, several loops which surround the active site are disordered, resulting in an open, solvent-exposed pocket. In the TEAS-(3) complex, as in the previously examined TEAS-farnesyl hydroxyphosphonate complex (PDB code 5EAT), the active site is in a closed conformation, as evidenced by the strong electron density apparent for residues in the A/C and J/K loops (FIG. 3). This closed conformation , which sequesters the hydrophobic active site from solvent, is probably adopted by the enzyme during catalysis. However, another active-site flanking loop (residues 449-476), which is ordered in all previously examined TEAS structures (Starks et al., 1997), is disordered in the TEAS-(3) complex.

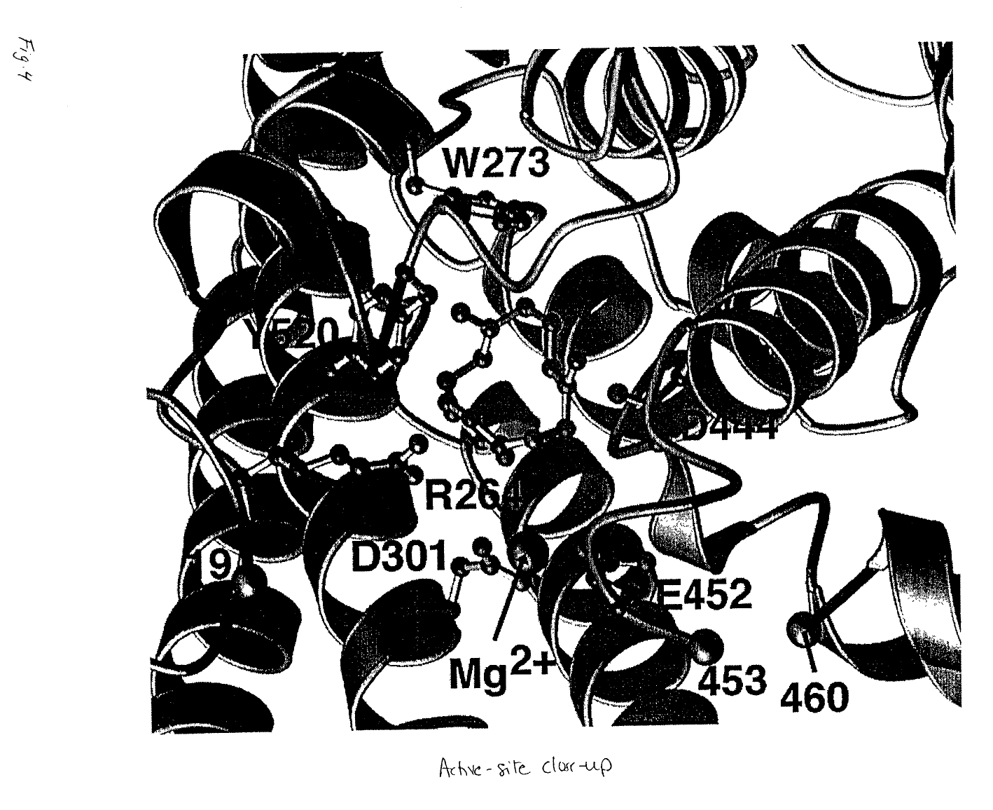

Of the three Mg²⁺ binding sites previously observed in TEAS (Starks et al., 1997), only site A appears to be fully occupied in the TEAS-(3) complex. No electron density is visible in the Mg²⁺ site B, which exhibits strong density in both uncomplexed TEAS (PDB code 5EAS) and the TEAS-farnesyl hydroxyphosphonate complex. Glu-452, one of the residues that coordinates this Mg²⁺ in 5EAS and 5EAT, is displaced slightly in the TEAS-(3) complex (FIG. 4). In addition, other residues near this site are disordered. Residues 454-459 do not exhibit sufficient electron density to allow them to be modeled, while residues 449-453 and 460-475 have high temperature factors (average 76). It is possible that occupation of Mg²⁺ site B facilitates ordering of this region. Mg²⁺ site C, which becomes occupied on FHP binding, exhibits weak electron density in the TEAS-(3) complex, suggesting that it may be partially occupied. Additional weak density is found at the active site entrance and may correspond to rapidly exchanging water molecules, a small population of dissociated pyrophosphate, or the pyrophosphate moiety of a small population of unreacted AGPP molecules bound at the active site. It is possible that the Mg²⁺ site C is occupied in those protein molecules with a bound pyrophosphate.

In the proposed macrocyclic alkaloid product (3), the carbon initially bonded to the pyrophosphate moiety is located at the back of the TEAS active site pocket, distal to the active site entrance. This orientation is opposite to that expected for a typical terpene cyclization reaction in which the substrate pyrophosphate moiety is coordinated to the magnesium ions and positively charged residues located at the active site entrance. To test the validity of this binding mode, other conformations and orientations of (3) were modeled in the active site electron density of the initial 3f₀-2F₀ difference electron density map. Conformation (3a) fit the electron density moderately well, and its positions and temperature factors were refined against the diffraction data. The resulting model had higher temperature factors for the cyclic product (Table 1), and subsequent electron density maps calculated using this model exhibited poorer quality electron density in the active site. This suggested that the initially modeled orientation was correct. In the initial orientation, the most polar group of (3), its nitrogen atom, is near the active site entrance, where it is able to hydrogen bond with water molecules or any residual bound pyrophosphate. The rest of the hydrophobic macrocycle can interact favorably with the hydrophobic active site surface. For example, the phenyl ring of (3) packs against the aromatic face of Tyr527. It is therefore likely that AGPP initially binds in the TEAS active site with its diphosphate moiety coordinated near the active site entrance. Following cyclization, the product adopts its most energetically favorable binding orientation.

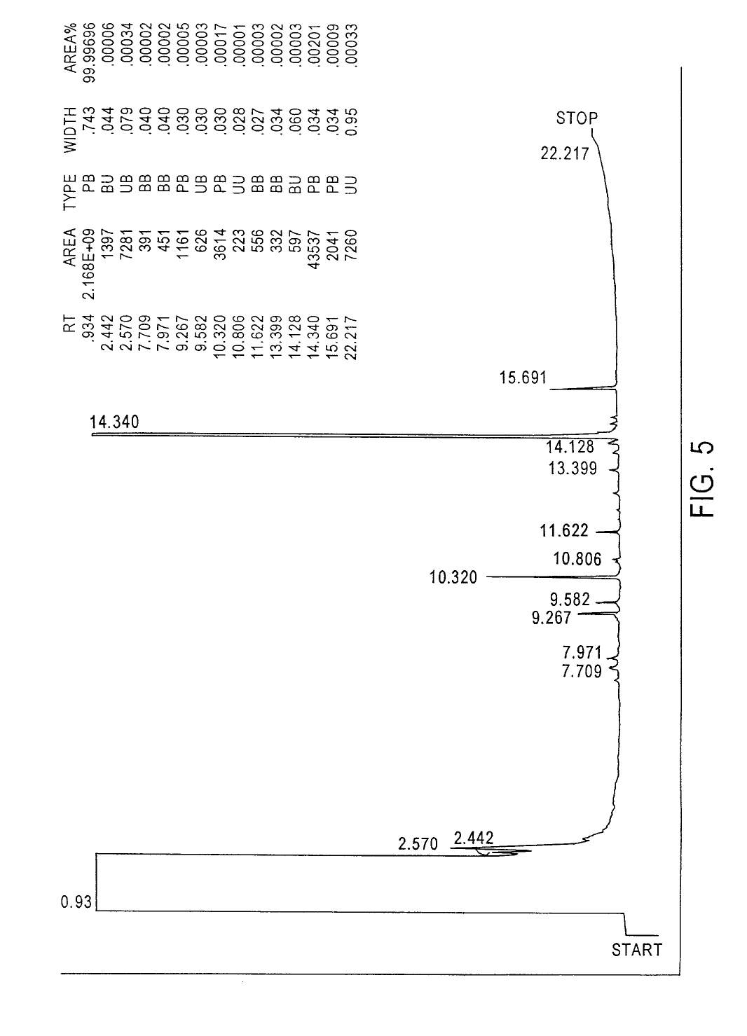

Gas Chromatographic and Mass Spectrometric Analysis of the Hexane-Extractable Product of TEAS Incubation with AGPP. In preliminary experiments, hexane, ethyl acetate, or ether extractions of reactions containing TEAS and [³H]AGPP yielded radioactivity in the organic phase, indicating the possibility of TEAS-catalyzed production of a predominantly hydrophobic product from AGPP. These data, in addition to the crystallographic evidence for TEAS-catalyzation of AGPP, led to efforts to elucidate the nature of the putative product through gas chromatographic and mass spectrometric analyses. Gas chromatography of hexane extracts from reactions containing AGPP and TEAS indicated the formation of a product with a retention time of 14.3 min. (FIG. 5). This reaction was distinguishable from TEAS-catalyzed formation of 5-epi-aristolochene, as well as from a standard sample of AGOH (retention times of 9.5 and 17.0 min., respectively). When reactions containing TEAS and AGPP were incubated at 30° C., approximately 5-fold more product was obtained in comparison to reactions incubated on ice for an equivalent period of time. Control reactions containing AGPP but no enzyme showed no product formation after prolonged incubation at 40° C.

GC analyses were conducted on reaction extracts collected at zero time in an effort to verify that the putative product observed upon incubation of TEAS with AGPP was not an artifact present in the initial reaction mixture. Reactions contained AGPP and/or TEAS, or neither. Prior to initiation with enzyme, putative substrate, or water, reactions were extracted with hexane. Following initiation, reactions were re-vortexed immediately and the extraction was completed as described above. A GC peak at 14.3 min. was observed only in the hexane extract derived from the reaction containing both TEAS and AGPP, indicating that this peak is probably the result of TEAS catalysis. However, the detection of product in this reaction was unexpected, given the short reaction time (<2 min.) and the non-optimal conditions. When more stringent pre-quenching conditions (addition of 0.2 M KOH, 0.1 M EDTA, followed by extraction with hexane; Mathis et al., 1977) were used on an assay mixture containing TEAS, no reaction product was observed following initiation of the reaction with AGPP.

A more rigorous verification that the GC peak at 14.3 min. was the result of TEAS catalysis was obtained by exploiting the dependence of TEAS activity on MgCl₂. Biochemical and crystallographic evidence indicate that Mg²⁺ is a required cofactor in TEAS catalysis due to its role in coordinating the pyrophosphate moiety of FPP, thereby facilitating both the formation of a catalytically competent enzyme-substrate complex as well as the ionization of pyrophosphate from the substrate (Vögeli et al. 1990, Starks et al., 1997). If formation of the putative cyclized product form AGPP is the result of binding and catalysis within the TEAS active site, Mg²⁺ is expected to function in a similar capacity in this reaction. As predicted, the product observed in a reaction mixture containing TEAS, AGPP and 40 mM MgCl₂, was undetectable in a parallel reaction in which 10 mM EDTA was substituted for MgCl₂. Thus, formation of the putative cyclized product from AGPP is a Mg²⁺-dependent process that is catalyzed at the TEAS active site.

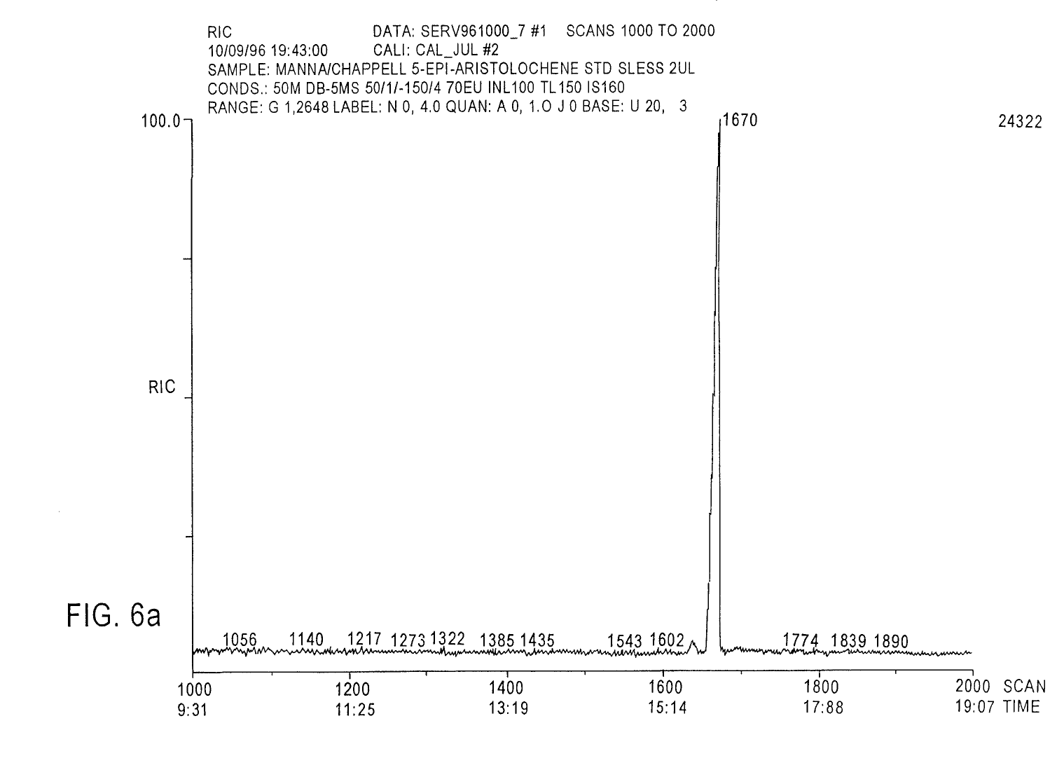

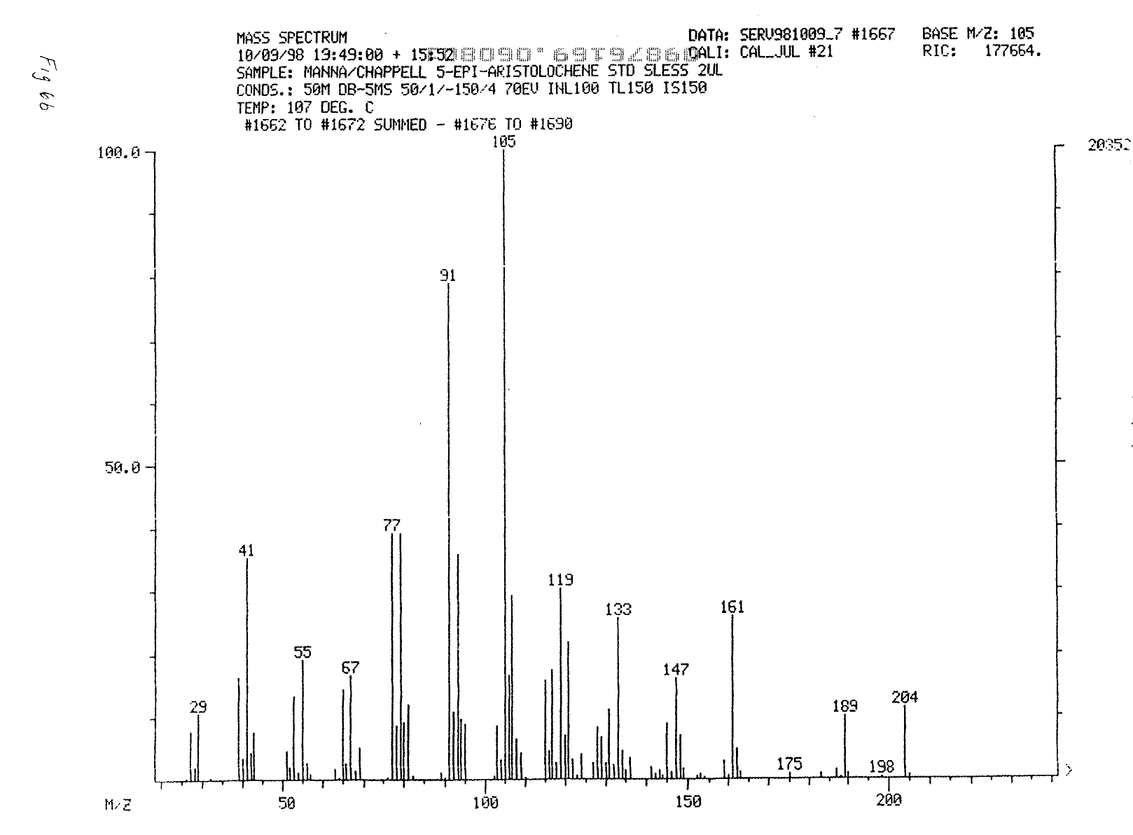

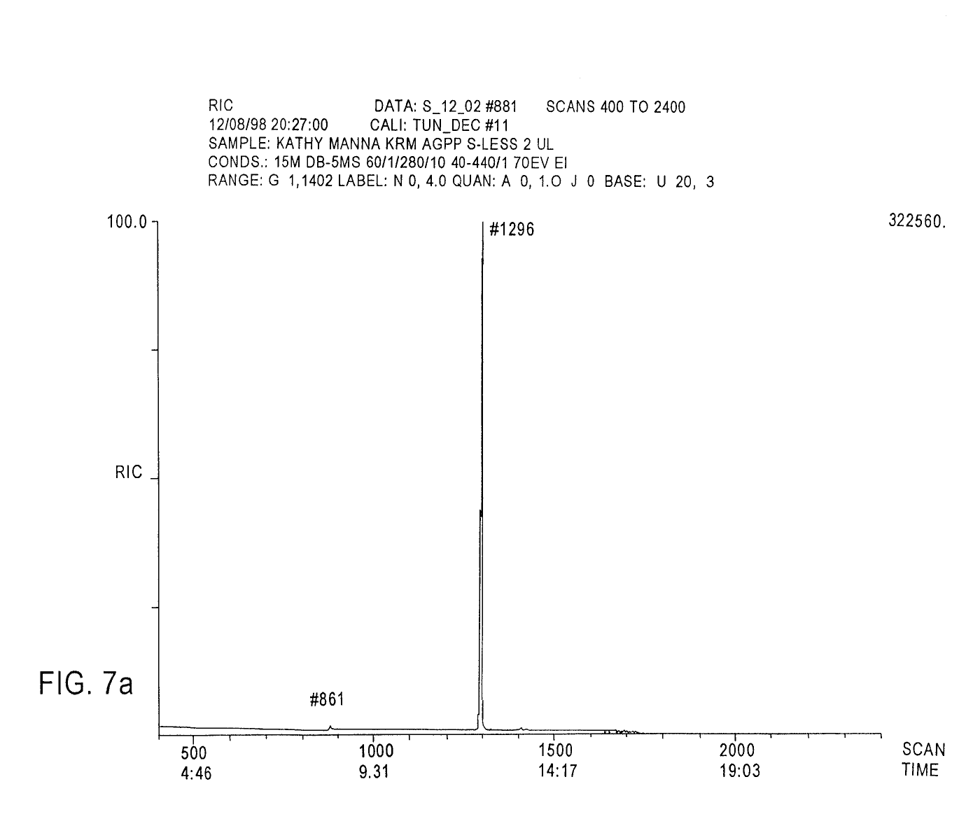

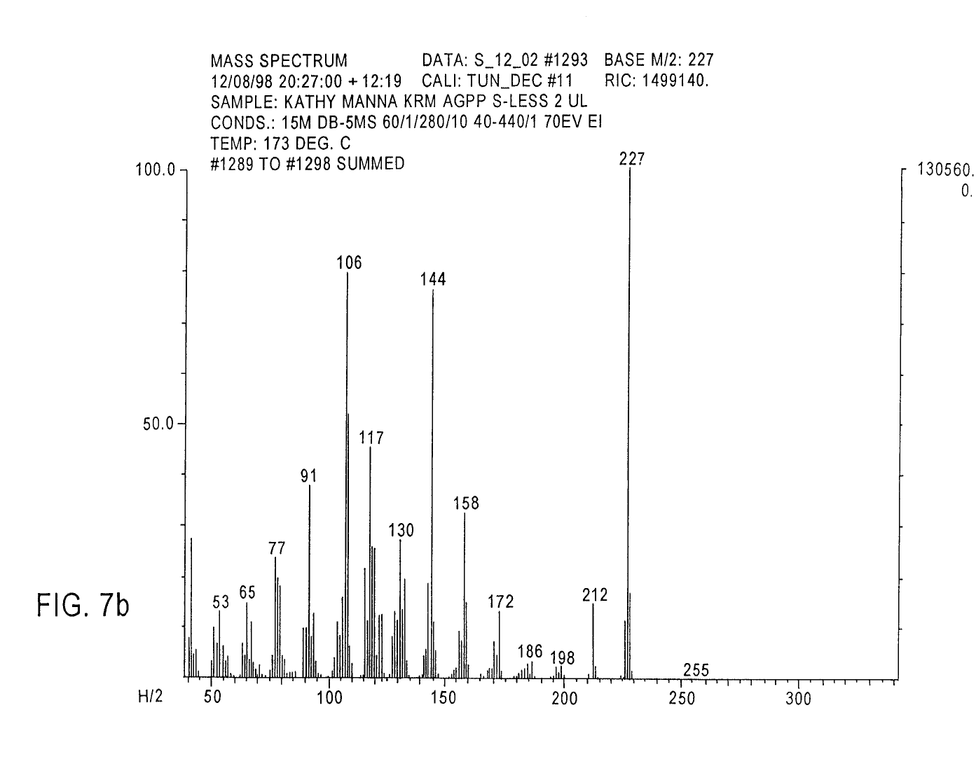

GC-MS and High Resolution MS Analyses of Hexane-Extractable Product of TEAS Incubation with AGPP. The exact mass, and corresponding molecular formula, for the putative product of TEAS-catalyzed AGPP cyclization were deduced through GC-MS and high resolution MS experiments. Although non-identical conditions were used in the gas chromatography of the 5-epi-aristolochene standard (FIG. 6a) and the reaction extracts (FIG. 7a), it is notable that each sample consisted of a pure compound and produced a single, distinct peak. Retention times for the putative cyclized product and the 5-epi-aristolochene standard were 12.3 and 15.9 min., respectively. The mass spectrum for the 5-epi-aristolochene standard (FIG. 6b) showed the expected molecular ion at 204 Da, as well as a fragmentation pattern corresponding to data published for the compound (Whitehead et al., 1989). The mass spectrum for the hexane-extractable product of AGPP (FIG. 7b) incubation with TEAS was characterized by a molecular ion at 227 Da and a fragmentation pattern distinctly different from that of 5-epi-aristolochene. This result confirmed that the proposed product of AGPP cyclization is distinct from the natural product of TEAS catalysis. The observed mass of 227 Da is consistent with the formula proposed for the putative cyclized product C₁₆H₂₁N. The accurate monoisotopic mass calculated for C₁₆H₂₁N is 227.1674 Da (C=12.0000 Da, H=1.00783 Da, N=14.0031 Da; Dr. Pyrek's calculation; I get 227.1675 Da). A mass of 227.1676 Da was determined by high resolution MS experiments and confirms the atomic composition of the macrocyclic alkaloid formed from TEAS-catalyzed cyclization of AGPP.

The synthesis of inventive compound (3) is shown in Scheme 2, below

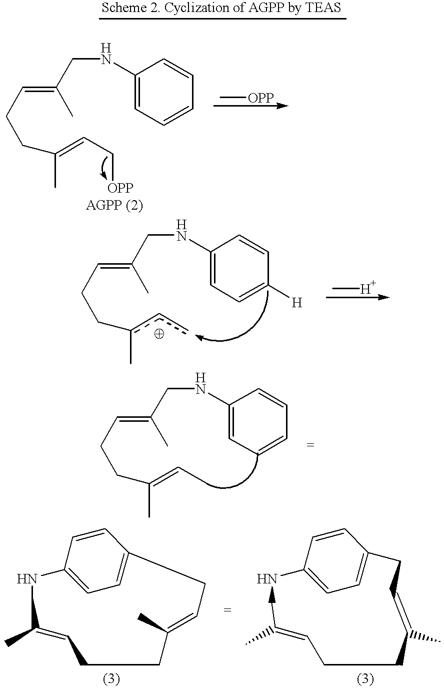

### Potential Applications of TEAS-Catalyzed Cyclization of Substrate Analogs

Compounds according to the present invention are suitable as inhibitors of Tobacco 5-epi-aristolochene synthase, and may be used as assay reagents therefor. The compounds may also be used for a number of other uses, such as agricultural, industrial and medicinal purposes. Potential agricultural uses include fungicide, insecticide, acaricide, nematocide, and herbicidal uses. The sulfonamide derivatives of (3), in particular, are of interest as insecticides and fungicides.

While the present invention has been described with reference to particular embodiments, the person having skill in the art will recognize that the methodologies herein employed may be modified or extended without departing from the general scope of the present invention. All references and public documents cited herein are specifically incorporated herein by reference.

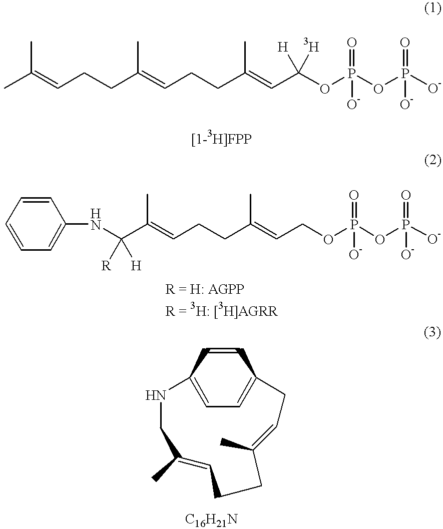

TABLE 1
(X-ray Diffraction Data)

| Data Collection Statistics | |
|---|---|
| Space Group | P4₁2₁2 |
| Unit Cell dimensions (Å) | 126.5 × 122.8 |
| Highest Resolution (Å) | 2.28 |
| Completeness (%) | |
| Overall (99-2.8 Å) | 89.6 |
| Highest Resolution Shell | 78.2 |
| Lowest Resolution Shell | 84.2 |
| I/σ | |
| Overall | 12.8 |
| Highest Resolution Shell | 2.2 |
| Rsym (%) | |
| Overall | 5.5 |
| Highest Resolution Shell | 58.7 |
| Mosaicity (*) | 0.6-0.9 |
| Data Refinement Statistics | |
| Resolution (Å) | 50-2.3 |
| Model Includes | |
| Total non-hydrogen atoms | 4404 |
| Water Molecules | 152 |
| Mg²⁺ ions | 1 |
| molecules | 1 |
| R (%, all data) | 25.1 |
| Rfree (%, all data) | 26.7 |
| Temperature Factors | |
| Main Chain | 48.7 |
| Side Chains and Water | 50.9 |
| (2) | 54.1 |
| (2), conformation a | 59.3 |
| Overall | 49.8 |
| Predicted by Wilson Plot | 34.1 |
| R.m.s. deviations from ideality | |
| Bond lengths (Å) | 0.18 |
| Bond angles (*) | 1.5 |
| Dihedral angles | 19.0 |

Additional objects, advantages and other features of the invention will be set forth in the description that follows, and in part will become apparent to those having skill in the art upon consideration of the following description and appended figures, or upon practice of the disclosed invention. The objects and advantages of the invention may be realized and obtained as particularly pointed out in the appended claims.

The purpose of the above description and examples is to illustrate some embodiments of the present invention without implying any limitation. It will be apparent to those of skill in the art that various modifications and variations may be made to the systems, devices and methods of the present invention without departing from the spirit or scope of the invention. All patents and articles cited herein are specifically incorporated herein in their entireties.

Acknowledgements:

Professor Jan St. Pyrek for mass spec. experiments

University of Kentucky, Mass Spectrometry Facility

References:

Back, K., Yin, S. & Chappell, J. (1994) Expression of a Plant Sesquiterpene Cyclase Gene in Escherichia coli, Arch. Biochem. Biophys. 315, 527-532.

Bradford, M M. (1976), Anal. Biochem., 72, 248.

Mathis, J. R., Back, K., Starks, C., Noel, J., Poulter, C. D. & Chappell, J. (1997) Pre-Steady State Study of Recombinant Sesquiterpene Synthase, Science, 36, 8340-8348.

Starks, C. M., Back, K., Chappell, J. & Noel, J. P. (1997), Structural Basis for Cyclic Terpene Biosynthesis by Tobacco 5-Epi-Aristolochene Synthase, Science, 277, 1815-1820.

Vögeli, U. & Chappell, J. (1988), Introduction of Sesquiterpene Cyclase and Suppression of Squalene Synthetase Activities in Plant Cell Cultures Treated with Fungal Elicitor, Plant Physiol., 88, 1291-1296.

Vögeli, U., Freeman, J. W. & Chappell, J. (1990), Purification and Characterization of an Inducible Sesquiterpene Cyclase from Elicitor-Treated Tobacco Cell Suspension Cultures, Plant Physiol., 93, 182-187.

Whitehead, I. M., Threlfall, D. R. & Ewing, D. F. (1989), 5-Epi-Aristolochene is a Common Precursor of the Sesquiterpenoid Phytoalexins Capsidiol and Debneyol, Phytochemistry, 775-779.

We claim:

1. A compound according to the formula: 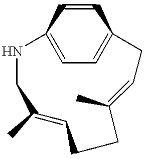
or a derivative, or salt thereof.

2. The compound according to claim 1, wherein the salt is an organic acid or inorganic acid salt thereof.

3. The compound according to claim 1, wherein the derivative is an amide or sulfonamide derivative thereof.

4. A process of making a compound of the formula: 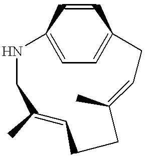
said process comprising: cyclizing a 8-anilino-geranyl pyrophosphate in the presence of a suitable enzyme.

5. The process according to claim 4, wherein the enzyme is an aristolochene synthase.

6. The process according to claim 5, wherein the enzyme is a plant-derived aristolochene synthase.

7. The process according to claim 6, wherein the enzyme is Tobacco 5-epi-aristolochene synthase (TEAS).

8. The process according to claim 4, wherein the process further comprises a step of preparing a derivative or acid-addition salt of the compound.

9. The process according to claim 4, wherein the process further comprises a step of preparing an acid-addition salt, which step comprises adding to the compound sufficient organic or inorganic acid to make said acid-addition salt.

10. The process according to claim 4, wherein the process further comprises a step of preparing an amide of the compound, which step comprises adding to the compound an amount of an organic acid under conditions suitable to form said amide.

11. The process according to claim 4, wherein the process further comprises a step of preparing a sulfonamide of the compound , which step comprises adding to the compound an amount of a sulfonic acid under conditions suitable to form said sulfonamide.

12. A process of manufacturing a bicyclic compound of the formula: 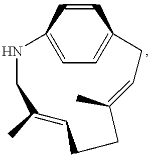
, the process comprising dissolving 8-anilino-geranyl pyrophosphate in a suitable solvent to produce a solution and contacting said solution with a means for cyclizing said AGPP to form the bicyclic compound.
